(19) United States
(12) Patent Application Publication
(10) Pub. No.: US 2015/0031100 A1
(43) Pub. Date: Jan. 29, 2015

(54) COMPOSITIONS AND METHODS FOR CHEMICAL REPORTER VECTORS

(71) Applicant: THE REGENTS OF THE UNIVERSITY OF COLORADO, A BODY CORPORATE, Denver, CO (US)

(72) Inventors: Ryan T. Gill, Denver, CO (US); Michael D. Lynch, Westminster, CO (US); Tirzah Y. Mills, Boulder, CO (US)

(73) Assignee: THE REGENTS OF THE UNIVERSITY OF COLORADO, A BODY CORPORATE, Denver, CO (US)

(21) Appl. No.: 14/192,554

(22) Filed: Feb. 27, 2014

Related U.S. Application Data
(63) Continuation of application No. 11/833,879, filed on Aug. 3, 2007, now abandoned.
(60) Provisional application No. 60/836,481, filed on Aug. 8, 2006.

(51) Int. Cl. C12N 15/10; C12P 7/46

(52) U.S. Cl. CPC C12N 15/1086; C12P 7/46
USPC 435/145; 506/10; 435/252.33; 506/14

(57) ABSTRACT

Embodiments of the present invention concerns methods and compositions for the construction of a series of vectors containing a chemical sensing module to assess the production of a chemical compound by a microorganism.

### CROSS-REFERENCE TO RELATED APPLICATIONS

This application claims benefit under 35 U.S.C. §119(e) of provisional U.S. Application No. 60/836,461, filed Aug. 8, 2006 and is hereby incorporated herein by reference in its entirety for all purposes.

### FEDERALLY FUNDED RESEARCH

The studies disclosed herein were supported in part by grants R21 AI055773-01 and F31 AI056687 from the National Institutes of Health and BES0228584 from the National Science Foundation. The U.S. Government may have certain rights to practice the subject invention.

### FIELD

This application relates generally to methods, compositions and uses of vectors for assessing genetic alterations and/or genetic control. This application also relates generally to methods, compositions and uses of vectors for assessing the production of organic molecules by a microorganism. In one particular embodiment, the present invention relates to compositions and methods of generating vectors for rapidly assessing the production of an organic molecule by an organism such as a bacteria. In another embodiment, the present invention relates to compositions and methods of generating vectors for rapidly assessing the production of, for example, 1,4 succinic, fumaric and malic acids, 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, and xylitol/arabinitol, and lactate by a microorganism, such as a bacteria.

### BACKGROUND

Oil costs have risen dramatically over the past several years. Most experts now believe that such cost increases will continue and that oil production capacity will peak in the near future. Alternative sources of inexpensive materials and energy for the production of fuels and other chemicals most be developed.

Biorefining seeks to develop renewable sources, such as agricultural or municipal waste, for such purposes. The basic model involves the conversion of waste material (i.e., corn) into sugars (i.e. hexoses, pentoses) that can be fermented by engineered organisms to produce value added products such as fuels (i.e. ethanol or hydrogen) or commodity chemicals (i.e. monomers/polymers). While much debate still exists regarding the long term commercial viability of ethanol as a gasoline replacement, biological routes for the production of commodity chemicals have been proven as economically attractive alternatives to conventional petrochemical routes. As one example, a decade long DuPont/Genencor collaboration was so successful that DuPont has now invested in the development of 800,000 liters E. coli based process for the production of 1,3 propanediol (an estimated $5-8 billion/year product).

Organic acids represent an important platform of future biorefining chemicals. In a report recently released by the National Renewable Energy Laboratory, eight different organic-acids were ranked among the top 12 highest priority biorefining chemicals. These 12 building block chemicals can be converted to high-value, bio-based chemicals or materials for use in industry. In addition to these chemicals, fuels such as ethanol, butanol, and hexanol can be produced from biomass.

Succinate is a natural product of microbial metabolism, which has led to much activity directed at the development of biological routes to succinate production. Several different genetic strategies have been investigated for the production of succinate in E. coli, which is an attractive host organism because of its large nutrient source range (i.e. pentoses), fast growth, and ability to be easily genetically modified when compared to alternative organisms. Although production of high levels (100 g/L) of succinate have been demonstrated, resultant strains are either poor growers or are limited to aerobic conditions which are not as attractive from a commercial standpoint. A need currently exists for improving the identification of high producing strains and increasing productivity of organic acid producing strains of bacteria.

### SUMMARY OF THE INVENTION

Some embodiments herein concern vector compositions having a chemical sensor module. In accordance with these embodiments a vector composition can include a) a chemical sensor module; b) a regulated promoter; and c) a selectable marker. In certain more particular embodiments, the vector can be a plasmid. In other embodiments, a chemical sensor module senses an organic compound. Exemplary organic compounds can include, but are not limited to malate, lactate, succinate and pyruvate. In other examples, a chemical sensor module can regulate expression from the regulated promoter in response to the presence of the organic compound.

In yet other embodiments, methods contemplated herein may include a) generating a vector having a selectable marker, a chemical sensor module and a regulated promoter; b) using the vector to assess the survivability of a bacterial strain; and c) selecting a bacterial strain based on survivability, wherein the survivability is indicative of a genetic modification of the strain. In a particular method, survivability of the strain to genetically select for improved survival of the strain can be assessed. In one example, a method herein can include identifying one or more changes in the strain that increase its survivability. In other embodiments, the changes can include one or more genetic mutations.

Some embodiments herein concern methods for enhanced production of a compound in a host cell including, but not limited to, a. optimizing a single expression regulatory sequence for producing a compound in the host cell utilizing at least two or more nucleic acid constructs; b. mobilizing the single expression regulatory sequence into a chemical sensor nucleic acid construct and transforming the host cell with the chemical sensor nucleic acid construct; c. screening for enhanced production of the compound in the host cell by providing a genomic library of predetermined sized genomic sequences, wherein the sequences are transformed in the host cell and one or more of the sequences are associated with the enhanced production; d. determining the host cell in (c) that provides the enhanced production; and e. culturing the host cell in (d) to provide the compound. In accordance with these methods, the nucleic acid construct(s) can be episomal or integrated into the host cell's genome.

Other embodiments herein concern, a host cell comprising a nucleic acid construct comprising an expression regulatory element optimized for production of a compound in a host cell and a second expression regulatory element comprising a chemical sensor module, wherein the chemical sensor module is responsive to the compound.

Yet other embodiments concern a population of host cells including, but not limited to, a nucleic acid construct including an expression regulatory element optimized for production of a compound in a host cell, and a second nucleic acid construct including a chemical sensor module, wherein the chemical sensor module is responsive to the compound, and wherein the population includes a genomic library of predetermined sized sequences.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1. illustrates an exemplary schematic of a SCALEs (multi-Scale Analysis of Library Enrichments) analysis.

FIG. 2. illustrates a schematic of production of a compound and compound reporter imputing antibiotic resistance on a microorganism.

FIG. 3. illustrates a representative pSUC plasmid.

FIG. 4. illustrates an exemplary histogram of increased resistance to chloramphenicol due to increased production of an organic compound, succinate.

FIG. 5. illustrates an exemplary histogram of increased resistance to trimethoprim and increased production of an organic compound, succinate.

FIG. 6. illustrates an exemplary histogram of survival in anaerobic conditions of E. coli NZN111 in the presence or absence of reporter plasmids.

FIG. 7. illustrates an exemplary histogram of clones from a library (in NZN111+pSUC-TMP) plated and grown under succinate producing fermentative anaerobic conditions for 12 hours and then allowed to recover aerobically for 12 hrs.

FIG. 8. illustrates an exemplary histogram of clones from a library (in NZN111+pSUC-TMP) plated and grown under succinate producing fermentative anaerobic conditions for 12 hours and then allowed to recover aerobically for 24 hrs.

### DEFINITIONS

Terms that are not otherwise defined herein are used in accordance with their plain and ordinary meaning.

As used herein, “a” or “an” may mean one or more than one of an item.

### DESCRIPTION OF ILLUSTRATIVE EMBODIMENTS

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation.

In some cases, well known methods or components have not been included in the description, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

One embodiment of the present invention concerns biorefining, biomass (crops, trees, grasses, crop residues, forest residues, etc.) and using biological conversion, fermentation, chemical conversion and catalysts Biomass to generate and use organic compounds. These organic compounds can then subsequently be converted to valuable derivative chemicals. However, the organic acids are toxic by nature and thus inhibitory to the production organisms at low levels. In order to optimize production of the organic acid intermediates, engineering tolerance to the organic acid may be a factor, permitting increased levels of production. Since commodity chemicals exist in a competitive environment, optimization might be necessary for the economic feasibility of biorefining. Therefore, compositions and methods disclosed herein are directed toward identifying and maximizing bacterial strains that produce increased quantities of organic compounds for use in bioproduction products and systems.

One objective of the disclosed embodiments is to demonstrate the commercial potential of a novel strain engineering tool. One problem in strain engineering efforts concerns how to increase the throughput of screening assays that are directed at identifying mutants with increased product synthesis rates. One approach was directed to tying product levels to cell survival or growth rate. For example, enormous libraries of mutants can be grown in competition and those that either survive or are most enriched will exhibit increased levels of the relevant product. This is one approach to strain engineering. However, it is very difficult to construct a strategy that links product levels with survival. Specific approaches have been reported, and such approaches were of high academic and commercial impact, however, no strategy has proven to be generally applicable.

In various embodiments, growth selections can be used to allow for evaluation of billions of cells simultaneously (for example: selecting organisms that produce large quantities of a particular chemical compound). Genetic screens often proceed one cell at a time. Screens are used to detect individual compounds. Selections are tied to growth. Therefore, in one particular embodiment, bacterial organisms containing one or more of the vectors of the disclosed methods can be used to select organisms based on growth in a selective environment.

Disclosed herein are technologies that can be used to bridge gaps in the development of new genetic strategies for the production of chemical compounds. These novel high-throughput strategies allow evaluation of enormous numbers of different genetic modifications for any effects on chemical production such as the production of organic compounds including, but not limited to, succinate, fumaric and malic acids, 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, and lactate. In certain embodiments, these strategies will be used to identify a variety of different genetic methods such as i) improved production of succinate anaerobically but the production can be improved by the disclosed methods and ii) production is anaerobic, and thus will either identify the same mechanisms employed in the previously reported aerobic strains or will represent a minimum and non-obvious use of such mechanisms in anaerobic organism production. In addition, these vectors may be used in conjunction with the SCALEs technology, (Provisional Application No. 60/611,377 filed Sep. 20, 2004 and U.S. patent application Ser. No. 11/231,018 filed Sep. 20, 2005, both entitled: “Mixed-Library Parallel Gene Mapping Quantitation Microarray Technique for Genome Wide Identification of Trait Conferring Genes” incorporated herein by reference in their entirety), as genetic alterations of organisms and for genetic selection strategies.

In some embodiments herein, a selectable markers includes, but are not limited to antibiotic resistance cassettes, including but not limited to resistance to chloramphenicol, tetracycline, trimethoprim, kanamycin, beta-lactams, tellurite, amino acid synthesis genes in an auxotroph, or other genes necessary for growth in a particular environment or media. In certain embodiments, a promoter may be used as a genetic element disclosed herein. Promoters can include, but are not limited to lac, ptrc, dcuBP, pbad, pdhrP, mtlP, rbsdP, glpTQp, srlRp, and gudPp.

### Bacterial Based Process for the Production

Production of organic molecules can be achieved biochemically through microorganisms. In one embodiment, the microorganism is a bacteria. In one particular embodiment, the bacteria is E. coli. In certain embodiments, strains with increased production rate can be engineered.

In one embodiment, a combination of newly developed population based genomics tools including the SCALEs (multi-SCale Analysis of Library Enrichment) approach recited previously and selection strategies have recently made the mapping of fitness to genotype a high throughput endeavor. However, when these tools are applied to non-selectable phenotypes, such as metabolite or organic acid production, an enormous amount of screening is required. Screening populations with greater than 107 members becomes prohibitive and as in all screening strategies is a severe limitation when compared to selection. For example, a limitation of the SCALEs approach is only selectable phenotypes can be analyzed. It was desired to extend this approach to also include non-selectable phenotypes.

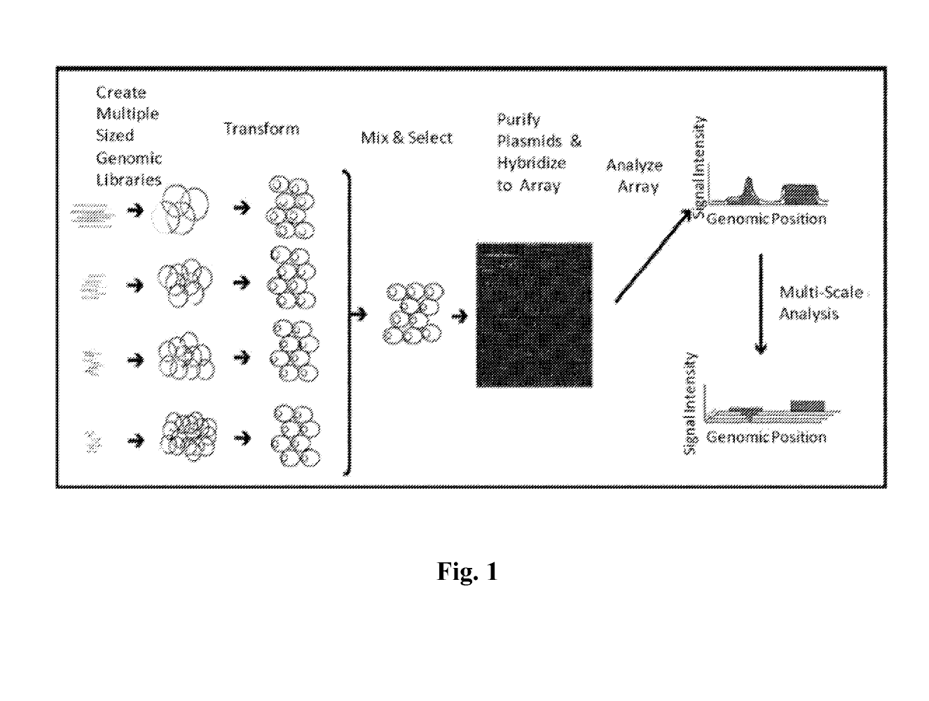

Conventional methods utilize screening tools to test how much product each clone or variant may make. This is time intensive. A selective process would allow for variants or clones that make more product to grow faster or survive in an environment. Variants producing less product would die, thus greatly reducing the number of variants needing testing. In one embodiment herein, methods are disclosed for selecting for genomic library clones that exhibited altered product accumulation levels. The two objectives of this study were to 1) construct a reporter (pSUC) that will confer a selectable phenotype in response to a model product (e.g. succinate) and 2) develop a selection combining the reporter and SCALEs which is termed ‘ReSCALEs’ (Reporter linked SCALEs). In certain embodiments herein, a reporter plasmid can be introduced to a cell. If an increased level of product is achieved, the reporter plasmid will render the cell resistant to an antibiotic present in the culture media (see FIG. 1). Then multiple sized libraries, as per the SCALEs method, are introduced into this cell. If the library plasmids increase product formation, the reporter vector will render the cell resistant to an antibiotic and only cells harboring library plasmids that increase product formation will survive. In accordance with these embodiments, genes on these library plasmids that enable increased production rates can be identified.

In one embodiment, an organic compound reporter vector can be designed which, in the presence of the organic compound, confers increased tolerance for a selective condition normally sensitive to a bacterial organism. In a more particular embodiment, the reporter vector can be a succinate reporter vector. For example, a succinate reporter vector can be designed which in the presence of succinate confers for example, antibiotic resistance, to a normally sensitive bacteria. In one more particular embodiment, the bacteria is E. coli. One use of this exemplary vector is to engineer bacterial strains to increase production of succinate. This exemplary vector utilizes the expression of a naturally occurring two component regulatory system. In accordance with these embodiments, the expression, normally based on specific environmental conditions, can be made constitutive by placing these genes behind a constitutive promoter. These two proteins when expressed can respond to an organic compound, such as succinate, by turning on a gene from a particular promoter. In certain particular embodiments, the promoter is dcuBP2. In one exemplary method, an antibiotic resistance gene, such as the gene for kanamycin resistance behind that promoter links the presence of succinate to antibiotic resistance. In certain embodiments, a constitutive promoter (P1), and a compound responsive SP a succinate responsive promoter, DCUBp2 can be constructed. The reporter or KAN gene could be any antibiotic resistance gene or selectable marker gene.

Embodiments herein disclose technologies that can be used to develop new genetic strategies for increased production, tolerance and/or sensing of chemical compounds. In one embodiment, methods disclosed herein concern novel promoter selection strategies. These embodiments provide promoter selection for increased chemical production, such as, increased production of chemical compounds including but not limited to small molecules, a fermentation product, an alcohol ethanol, propanol, propionate, 3-hydroxypropionate, butanol, isobutanol, butyrate, succinate, maleate, malate, fumarate, pentanol, hexanol, octanol, glucarate, glutamate, itaconate, levulinate, hydroxybutyrolactone, glycerol, sorbitol, xylitol and a combination thereof. For example, these strategies can identify a variety of different genetic elements and methods that are commercially viable.

### Nucleic Acids

As described herein, an aspect of the present disclosure concerns isolated nucleic acids and methods of use of isolated nucleic acids. In certain embodiments, the nucleic acid sequences disclosed herein have utility as hybridization probes or amplification primers. These nucleic acids may be used, for example, in diagnostic evaluation of tissue samples. In certain embodiments, these probes and primers consist of oligonucleotide fragments. Such fragments should be of sufficient length to provide specific hybridization to a RNA or DNA tissue sample. The sequences typically will be 10-20 nucleotides, but may be longer. Longer sequences, e.g., 40, 50, 100, 500 and even up to full length, are preferred for certain embodiments.

Nucleic acid molecules having contiguous stretches of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75 80, 85, 90, 95, 100, 125, 150, 175, 200, 250, 300, 400, 500, 600, 750, 1000, 1500, 2000, 2500 or more nucleotides from a sequence selected from the disclosed nucleic acid sequences are contemplated. Molecules that are complementary to the above mentioned sequences and that bind to these sequences under high stringency conditions also are contemplated. These probes will be useful in a variety of hybridization embodiments, such as Southern and Northern blotting.

The use of a hybridization probe of between 14 and 100 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained. One will generally prefer to design nucleic acid molecules having stretches of 20 to 30 nucleotides, or even longer where desired. Such fragments may be readily prepared by, for example, directly synthesizing the fragment by chemical means or by introducing selected sequences into recombinant vectors for recombinant production.

Accordingly, the nucleotide sequences of the invention may be used for their ability to selectively form duplex molecules with complementary stretches of genes or RNAs or to provide primers for amplification of DNA or RNA from tissues. Depending on the application envisioned, one may desire to employ varying conditions of hybridization to achieve varying degrees of selectivity of probe towards target sequence.

For applications requiring high selectivity, one will typically desire to employ relatively stringent conditions to form the hybrids, e.g., one will select relatively low salt and/or high temperature conditions, such as provided by about 0.02 M to about 0.10 M NaCl at temperatures of about 50□C to about 70□C. Such high stringency conditions tolerate little, if any, mismatch between the probe and the template or target strand, and would be particularly suitable for isolating specific genes or detecting specific mRNA transcripts. It is generally appreciated that conditions can be rendered more stringent by the addition of increasing amounts of formamide.

For certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37□C to about 55□C, while a low stringency condition could be provided by about 0.15 M to about 0.9 M salt, at temperatures ranging from about 20□C to about 55□C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl2, 10 mM dithiothreitol, at temperatures between approximately 20° C. to about 37□C. Other hybridization conditions utilized could include approximately 10 mM Tris-HCl (pH 8.3), 50 mM KCl, 1.5 μM MgCl2, at temperatures ranging from approximately 40° C. to about 72° C.

In certain embodiments, it will be advantageous to employ nucleic acid sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known which can be employed to provide a detection means visible to the human eye or spectrophotometrically, to identify specific hybridization with complementary nucleic acid-containing samples.

In general, it is envisioned that the hybridization probes described herein will be useful both as reagents in solution hybridization, as in PCR, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface to remove non-specifically bound probe molecules, hybridization is detected, or even quantified, by means of the label.

It will be understood that this invention is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present invention are incorporated into vectors, such as plasmids disclosed herein, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

Where an expression product is to be generated, it is possible for the nucleic acid sequence to be varied while retaining the ability to encode the same product. Reference to the codon chart, provided above, will permit those of skill in the art to design any nucleic acid encoding for the product of a given nucleic acid.

### Plasmid Preparations

Plasmid preparations and replication means are well known in the art. See for example, U.S. Pat. Nos. 4,273,875 and 4,567,146 incorporated herein their entirety. Some embodiments of the present invention include providing a portion of genetic material of a target microorganism and inserting the portion of genetic material of a target microorganism into a plasmid for use as an internal control plasmid.

### Amplification

Embodiments of the present invention include providing conditions that facilitate amplification of at least a portion of a target genetic material. However, it should be appreciated that the amplification conditions of embodiments of the present invention are not necessarily 100% specific.

The embodiments of the present invention include any method for amplifying at least a portion of a microorganism's genetic material (such as Polymerase Chain Reaction (PCR), Real-time PCR (RT-PCR), NASBA (nucleic acid sequence based amplification)). In one embodiment, Real time PCR (RT-PCR) can be a method for amplifying at least a portion of a target microorganism's genetic material while simultaneously amplifying an internal control plasmid for verification of the outcome of the amplification of a microorganism's genetic material.

While the scope of the present invention includes any method (for example, Polymerase Chain Reaction, i.e., PCR, and nucleic acid sequence based amplification, i.e., NASBA) for amplifying at least a portion of the microorganism's genetic material, for one example, the present invention describes embodiments in reference to PCR technique.

Amplification of a genetic material, e.g., DNA, is well known in the art. See, for example, U.S. Pat. Nos. 4,683,202, and 4,994,370, which are incorporated herein by reference in their entirety. Methods of the present invention include providing conditions that would allow co-amplification of an internal control plasmid's portion of a microorganism's genetic material and a portion of the microorganism's genetic material of a test sample, if the target microorganism is present in the sample and the conditions for the method support the amplification of the internal control plasmid. In this manner, detection of the amplification products by a specific probe for each product of the internal control plasmid's portion of a microorganism's genetic material and a portion of the microorganism's genetic material is indicative of the presence of the microorganism in the sample and that the conditions for the amplification are working. Thus, a negative result indicative of absence of a target microorganism can be confirmed.

Typically, to verify the working conditions of PCR techniques, positive and negative external controls are performed in parallel reactions to the sample tubes to test the reaction conditions, for example using a control nucleic acid sequence for amplification. In some embodiments of the present invention, an internal control can be used to determine if the conditions of the RT-PCR reaction is working in a specific tube for a specific target sample. Alternatively, in some embodiments of the present invention, an internal control can be used to determine if the conditions of the RT-PCR reaction are working in a specific tube at a specific time for a specific target microorganism sample. For example, an internal control in an RT-PCR reaction can be used to determine whether lack of detection of a target microorganism in a given sample is truly negative or a false negative. In this manner, lack of detection of an amplification product of a portion of a target microorganism's genetic material is indicative of the absence of the microorganism in the sample and this is confirmed when the internal control (such as an internal plasmid control) is amplified in the same reaction tube at the same time indicating the conditions were conducive for amplification.

By knowing the nucleotide sequences of the genetic material in a target microorganism and in an internal control, specific primer sequences can be designed. In one embodiment of the present invention, at least one primer of a primer pair used to amplify a portion of genomic material of a target microorganism is in common with one of the primers of a primer pair used to amplify a portion of genetic material of an internal control such as an internal control plasmid. In one embodiment of the present invention, the primer is about, but not limited to 5 to 50 oligonucleotides long, or preferably about 10 to 40 oligonucleotides long or more preferably about 10 to 30 oligonucleotides long. Suitable primer sequences can be readily synthesized by one skilled in the art or are readily available from third party providers such as BRL (New England Biolabs), etc. Other reagents, such as DNA polymerases and nucleotides, that are necessary for a nucleic acid sequence amplification such as PCR are also commercially available.

### Detection

The presence or absence of PCR amplification product can be detected by any of the techniques known to one skilled in the art. In one particular embodiment, methods of the present invention include detecting the presence or absence of the PCR amplification product using a probe that hybridizes to a particular genetic material of the microorganism. By designing the PCR primer sequence and the probe nucleotide sequence to hybridize different portions of the microorganism's genetic material, one can increase the accuracy and/or sensitivity of the methods disclosed herein.

While there are a variety of labelled probes available, such as radioactive and fluorescent labelled probes, in one particular embodiment, methods of the present invention use a fluorescence resonance energy transfer (FRET) labeled probe as internal hybridization probes. In one particular embodiment of the present invention, an internal hybridization probe is included in the PCR reaction mixture so that product detection occurs as the PCR amplification product is formed, thereby reducing post-PCR processing time. Roche Lightcycler PCR instrument (U.S. Pat. No. 6,174,670) or other real-time PCR instruments can be used in this embodiment of the invention, e.g., see U.S. Pat. No. 6,814,934. PCR amplification of a genetic material increases the sensitivity of methods of the present invention to 101 organisms or less in comparison to about 105 microorganisms that are required in standard ELISA methods. In some instances, real-time PCR amplification and detection significantly reduce the total assay time so that test results may be obtained in about 12 hours. Accordingly, methods of the present invention provide rapid and/or highly accurate results relative to the conventional methods and these results are verified by an internal control.

### Nucleic Acid Amplification

Nucleic acids used as a template for amplification is isolated from cells contained in the biological sample, according to standard methodologies. (Sambrook et al., 1989) The nucleic acid may be genomic DNA or fractionated or whole cell RNA. Where RNA is used, it may be desired to convert the RNA to a complementary cDNA. In one embodiment, the RNA is whole cell RNA and is used directly as the template for amplification.

Pairs of primers that selectively hybridize to nucleic acids corresponding to specific markers are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintilography of incorporated radiolabel or fluorescent label or even via a system using electrical or thermal impulse signals (Affymax technology; Bellus, 1994).

### Primers

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences may be employed. Primers may be provided in double-branded or single-stranded form, although the single-stranded form is preferred.

### Template Dependent Amplification Methods

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., 1990, each of which is incorporated herein by reference in its entirety.

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641 filed Dec. 21, 1990. Polymerase chain reaction methodologies are well known in the art. Other amplification methods are known in the art besides PCR such as LCR (ligase chain reaction), disclosed in European Application No. 320 308, incorporated herein by reference in its entirety.

In another embodiment, Qbeta Replicase, previously described, may also be used as still another amplification method in the present invention. In this method, a replicative sequence of RNA which has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention. Walker et al., Proc. Nat'l. Acad. Sci. USA 89:392-396 (1992), incorporated herein by reference in its entirety.

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases may be added as biotinylated derivatives for easy detection. A similar approach is used in SDA. Target specific sequences may also be detected using a cyclic probe reaction (CPR). In CPR, a probe having 3′ and 5′ sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Still other amplification methods known in the art may be used with the methods described herein.

Davey et al., European Application No. 329 822 (incorporated herein by reference in its entirely) disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence may be used by the appropriate RNA polymerase to make many RNA copies of the DNA. These copies may then re-enter the cycle leading to very swift amplification. With proper choice of enzymes, this amplification may be done isothermally without addition of enzymes at each cycle. Because of the cyclical nature of this process, the starting sequence may be chosen to be in the form of either DNA or RNA.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., Genomics 4:560 (1989), incorporated herein by reference in its entirety.

### Separation Methods

Following amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

### Identification Methods

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products may then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety.

In one embodiment, detection is by Southern blotting and hybridization with a labeled probe. The techniques involved in Southern blotting are well known to those of skill in the art and may be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

In general, prokaryotes used for cloning DNA sequences in constructing the vectors useful in the invention include for example, any gram negative bacteris such as E. coli strain K12. Other microbial strains which may be used include P. aeruginosa strain PAO1, and E. coli B strain. These examples are illustrative rather than limiting. Other example bacterial hosts for constructing a library include but are not limited to Aeromonas, Acetobacter, Agrobacterium, Alcaligenes, Azorizobium, Bartonella, Bordetella, Brucella, Burkholderia, Caulobacter, Escherichia, Erwinia, Hyphomicrobium, Methylobacillus, Methybacterium, Mehylophilus, Pseudomonus, Paracoccus, Rhizobium, Ralstonia, Rhodobacter, Salmonella, Vibrio and Xanthomonas.

Prokaryotic cells also can be used for expression. The aforementioned strains, as well as E. coli W3110 (F.sup.-, .lamda..sup.-, prototrophic, ATTC No. 27325), and other enterobacteriaceae such as Salmonella typhimurium or Serratia marcescans, and various pseudomonas species can be used.

In general, plasmid vectors containing promoters and control sequences which are derived from species compatible with the host cell are used with these hosts. The vector ordinarily carries a replication site as well as one or more marker sequences which are capable of providing phenotypic selection in transformed cells. For example, a PBBR1 replicon region which is useful in many Gram negative bacterial strains can be used in the present invention.

Promoters suitable for use with prokaryotic hosts illustratively include the .beta.-lactamase and lactose promoter systems (Chang et al., “Nature”, 275: 615 [1978]; and Goeddel et al., “Nature” 21: 544 [1979]), alkaline phosphatase, the tryptophan (trp) promoter system (Goeddel “Nucleic Acids Res.” 8: 4057 [1980] and EPO Appln. Publ. No. 36,776) and hybrid promoters such as the tac promoter (H. de Boer et al., “Proc. Natl. Acad. Sci. USA” 80: 21 25 [1983]). However, other functional bacterial promoters are suitable.

In another embodiment, expression vectors used in prokaryotic host cells may also contain sequences necessary for efficient translation of specific genes encoding specific mRNA sequences that can be expressed from any suitable promoter. This would necessitate incorporation of a promoter followed by ribosomal binding sites or a Shine-Dalgarno (S.D.) sequence operably linked to the DNA encoding the mRNA.

Construction of suitable vectors containing the desired coding and control sequences employ standard ligation techniques. Isolated plasmids or DNA fragments are cleaved, tailored, and religated in the form desired to form the plasmids required.

For analysis to confirm correct sequences in plasmids constructed, the ligation mixtures are used to transform a bacteria strain such as E. coli K12 and successful transformants selected by antibiotic resistance such as tetracycline where appropriate. Plasmids from the transformants are prepared, analyzed by restriction and/or sequenced.

Host cells can be transformed with expression vectors of this invention and cultured in conventional nutrient media modified as is appropriate for inducing promoters, selecting transformants or amplifying genes. The culture conditions, such as temperature, pH and the like, are those previously used with the host cell selected for expression, and will be apparent to the ordinarily skilled artisan.

“Transformation” refers to the taking up of an expression vector by a host cell whether or not any coding sequences are in fact expressed. Numerous methods of transfection are known to the ordinarily skilled artisan, for example, Ca salts.sub.4 and electroporation. Successful transformation is generally recognized when any indication of the operation of this vector occurs within the host cell.

In order to facilitate understanding of the following examples certain frequently occurring methods and/or terms will be described.

Digestion of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37 C. are ordinarily used, but may vary in accordance with the supplier's instructions. After digestion the reaction is electrophoresed directly on a polyacrylamide gel to isolate the desired fragment.

Recovery or isolation of a given fragment of DNA from a restriction digest means separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. This procedure is known generally (Lawn, R. et al., Nucleic Acids Res. 9: 6103 6114 [1981], and Goeddel, D. et al., Nucleic Acids Res. 8: 4057 [1980]).

Dephosphorylation refers to the removal of the terminal 5′ phosphates by treatment with bacterial alkaline phosphatase (BAP). This procedure prevents the two restriction cleaved ends of a DNA fragment from “circularizing” or forming a closed loop that would impede insertion of another DNA fragment at the restriction site. Procedures and reagents for dephosphorylation are conventional (Maniatis, T. et al., Molecular Cloning, 133 134 Cold Spring Harbor, [1982]). Reactions using BAP are carried out in 50 mM Tris at 68 C. to suppress the activity of any exonucleases which may be present in the enzyme preparations. Reactions are run for 1 hour. Following the reaction the DNA fragment is gel purified.

Ligation refers to the process of forming phosphodiester bonds between two double stranded nucleic acid fragments (Maniatis, T. et al., Id. at 146). Unless otherwise provided, ligation may be accomplished using known buffers and conditions with 10 units of T4 DNA ligase (“ligase”) per 0.5 .g of approximately equimolar amounts of the DNA fragments to be ligated.

Filling or blunting refers to the procedures by which the single stranded end in the cohesive terminus of a restriction enzyme-cleaved nucleic acid is converted to a double strand. This eliminates the cohesive terminus and forms a blunt end. This process is a versatile tool for converting a restriction cut end that may be cohesive with the ends created by only one or a few other restriction enzymes into a terminus compatible with any blunt-cutting restriction endonuclease or other filled cohesive terminus. In one embodiment, blunting is accomplished by incubating around 2 to 20 μg of the target DNA in 10 mM MgCl.sub.2, 1 mM dithiothreitol, 50 mM NaCl, 10 mM Tris (pH 7.5) buffer at about 37 C. in the presence of 8 units of the Klenow fragment of DNA polymerase 1 and 250 μM of each of the four deoxynucleoside triphosphates. The incubation generally is terminated after 30 min. phenol and chloroform extraction and ethanol precipitation

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (i.e. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications such as (a) an alternative linking group, (b) an analogous form of purine, (c) an analogous form of pyrimidine, or (d) an analogous sugar. For examples of analogous linking groups, purine, pyrimidines, and sugars see for example PCT publication No. WO 95/04064, which disclosure is hereby incorporated by reference in its entirety. Preferred modifications of the present invention include, but are not limited to, 5-fluorouracil, 5-bromouracil, 5-chlorouracil, 5-iodouracil, hypoxanthine, xantine, 4acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v) ybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2thiouracil, 3-(3-amino-3-N-2-carboxypropyl)uracil, and 2,6-diaminopurine. The polynucleotide sequences of the invention may be prepared by any known method, including synthetic, recombinant, ex vivo generation, or a combination thereof, as well as utilizing any purification methods known in the art. Methylenemethylimino linked oligonucleosides as well as mixed backbone compounds, may be prepared as described in U.S. Pat. Nos. 5,378,825; 5,386,023; 5,489,677; 5,602,240; and 5,610,289, which disclosures are hereby incorporated by reference in their entireties. Formacetal and thioformacetal linked oligonucleosides may be prepared as described in U.S. Pat. Nos. 5,264,562 and 5,264,564, which disclosures are hereby incorporated by reference in their entireties. Ethylene oxide linked oligonucleosides may be prepared as described in U.S. Pat. No. 5,223,618, which disclosure is hereby incorporated by reference in its entirety. Phosphinate oligonucleotides may be prepared as described in U.S. Pat. No. 5,508,279, which disclosure is hereby incorporated by reference in its entirety. Alkyl phosphonate oligonucleotides may be prepared as described in U.S. Pat. No. 4,469,863, which disclosure is hereby incorporated by reference in its entirety. 3′-Deoxy-3′-methylene phosphonate oligonucleotides may be prepared as described in U.S. Pat. Nos. 5,610,289 or 5,625,050 which disclosures are hereby incorporated by reference in their entireties. Phosphoramidite oligonucleotides may be prepared as described in U.S. Pat. Nos. 5,256,775 or 5,366,878 which disclosures are hereby incorporated by reference in their entireties. Alkylphosphonothioate oligonucleotides may be prepared as described in published PCT applications WO 94/17093 and WO 94/02499 which disclosures are hereby incorporated by reference in their entireties. 3′-Deoxy-3′-amino phosphoramidate oligonucleotides may be prepared as described in U.S. Pat. No. 5,476,925, which disclosure is hereby incorporated by reference in its entirety. Phosphotriester oligonucleotides may be prepared as described in U.S. Pat. No. 5,023,243, which disclosure is hereby incorporated by reference in its entirety. Borano phosphate oligonucleotides may be prepared as described in U.S. Pat. Nos. 5,130,302 and 5,177,198 which disclosures are hereby incorporated by reference in their entireties.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another by virtue of their sequence identities in a manner like that found in double-helical DNA with thymine or uracil residues linked to adenine residues by two hydrogen bonds and cytosine and guanine residues linked by three hydrogen bonds (See Stryer, 1995, which disclosure is hereby incorporated by reference in its entirety).

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance Creighton (1993); Seifter et al., (1990); Rattan et al., (1992)). Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) # of the number of nucleic acid inserts in the population of recombinant backbone molecules.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

In a specific embodiment, the polynucleotides of the invention are at least 15, 30, 50, 100, 125, 500, or 1000 continuous nucleotides. In another embodiment, the polynucleotides are less than or equal to 300 kb, 200 kb, 100 kb, 50 kb, 10 kb, 7.5 kb, 5 kb, 2.5 kb, 2 kb, 1.5 kb, or 1 kb in length. In a further embodiment, polynucleotides of the invention comprise a portion of the coding sequences, as disclosed herein, but do not comprise all or a portion of any intron. In another embodiment, the polynucleotides comprising coding sequences do not contain coding sequences of a genomic flanking gene (i.e., 5′ or 3′ to the gene of interest in the genome). In other embodiments, the polynucleotides of the invention do not contain the coding sequence of more than 1000, 500, 250, 100, 75, 50, 25, 20, 15, 10, 5, 4, 3, 2, or 1 naturally occurring genomic flanking gene(s).

Procedures used to detect the presence of nucleic acids capable of hybridizing to the detectable probe include well known techniques such as Southern blotting, Northern blotting, dot blotting, colony hybridization, and plaque hybridization. In some applications, the nucleic acid capable of hybridizing to the labeled probe may be cloned into vectors such as expression vectors, sequencing vectors, or in vitro transcription vectors to facilitate the characterization and expression of the hybridizing nucleic acids in the sample. For example, such techniques may be used to isolate and clone sequences in a genomic library or cDNA library which are capable of hybridizing to the detectable probe as described herein.

### Labels

Certain embodiments may involve incorporating a label into a probe, primer and/or target nucleic acid to facilitate its detection by a detection unit. A number of different labels may be used, such as Raman tags, fluorophores, chromophores, radioisotopes, enzymatic tags, antibodies, chemiluminescent, electroluminescent, affinity labels, etc. One of skill in the art will recognize that these and other label moieties not mentioned herein can be used in the disclosed methods.

Fluorescent labels of use may include, but are not limited to, Alexa 350, Alexa 430, AMCA (7-amino-4-methylcoumarin-3-acetic acid), BODIPY (5,7-dimethyl-4-bora-3a,4a-diaza-s-indacene-3-propionic acid) 630/650, BODIPY 650/665, BODIPY-FL (fluorescein), BODIPY-R6G (6-carboxyrhodamine), BODIPY-TMR (tetramethylrhodamine), BODIPY-TRX (Texas Red-X), Cascade Blue, Cy2 (cyanine), Cy3, Cy5,6-FAM (5-carboxyfluorescein), Fluorescein, 6-JOE (2′7′-dimethoxy-4′5′-dichloro-6-carboxyfluorescein), Oregon Green 488, Oregon Green 500, Oregon Green 514, Pacific Blue, Rhodamine Green, Rhodamine Red, ROX (6-carboxy-X-rhodamine), TAMRA (N,N,N′,N′-tetramethyl-6-carboxyrhodamine), Tetramethylrhodamine, and Texas Red. Fluorescent or luminescent labels can be obtained from standard commercial sources, such as Molecular Probes (Eugene, Oreg.).

Examples of enzymatic labels include urease, alkaline phosphatase or peroxidase. Colorimetric indicator substrates can be employed with such enzymes to provide a detection means visible to the human eye or spectrophotometrically. Radioisotopes of potential use include 14carbon, 3hydrogen, 125iodine, 32phosphorus and 35sulphur.

### Vectors for Gene Expression

In certain embodiments expression vectors are employed to assay the functional effects of certain sequences such as a bi-directional, host-factor independent transcriptional terminators sequence. Expression requires that appropriate signals be provided in the vectors, and which include various regulatory elements, such as enhancers/promoters from viral or mammalian sources that drive expression of the genes of interest in host cells. Bi-directional, host-factor independent transcriptional terminators elements may be incorporated into the expression vector and levels of transcription, translation, RNA stability or protein stability may be determined using standard techniques known in the art. The effect of the bi-directional, host-factor independent transcriptional terminators sequence may be determined by comparison to a control expression vector lacking the bi-directional, host-factor independent transcriptional terminators sequence, or to an expression vector containing a bi-directional, host-factor independent transcriptional terminators sequence of known effect.

### Regulatory Elements

The terms “expression construct” or “expression vector” are meant to include any type of genetic construct containing a nucleic acid coding for a gene product in which part or all of the nucleic acid coding sequence is capable of being transcribed. In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. The particular promoter employed to control the expression of a nucleic acid sequence of interest is not believed to be important, so long as it is capable of directing the expression of the nucleic acid in the targeted cell.

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed. Also contemplated as an element of the expression construct is a terminator. These elements can serve to enhance message levels and to minimize read through from the construct into other sequences.

### Reporter Genes

In certain embodiments of the invention, the expression construct will contain a reporter gene whose activity may be measured to determine the effect of a bi-directional, host-factor independent transcriptional terminators element or other element. Conveniently, the reporter gene produces a product that is easily assayed, such as a colored product, a fluorescent product or a luminescent product. Many examples of reporter genes are available, such as the genes encoding GFP (green fluorescent protein), CAT (chloramphenicol acetyltransferase), luciferase, GAL (β-galactosidase), GUS (β-glucuronidase), etc. The reporter gene employed is not believed to be important, so long as it is capable of being expressed and its level of expression may be assayed. Further examples of reporter genes are well known to one of skill in the art, and any such known gene may be used in the practice of the claimed methods.

### Kits

Is some embodiments, the present invention concerns kits for use with the methods described herein. The kits may comprise, in suitable container means, one or more vectors, each vector capable of being used in a broad range of bacteria. In various embodiments, such kits may contain additional components of use for the amplification, hybridization and/or detection of vector sequences and or inserts, which components may include but not limited to two or more amplification primers, buffer, nucleotides, labels (such as fluorescent labels), labeled primers, polymerase, enzymes, enzyme substrates, control probes, control amplification templates, molecular weight standards or any other kit component known in the art.

The kits may further include a suitably aliquoted composition of the probes and/or primers, whether labeled or unlabeled, as may be used to prepare a standard curve for a detection assay. The components of the kits may be packaged either in aqueous media or in lyophilized form.

The container means of the kits will generally include at least one vial, test tube, flask, bottle, syringe or other container means, into which the probes and/or primers may be placed, and preferably, suitably aliquoted. Where an additional component is provided, the kit will also generally contain additional containers into which this component may be placed. The kits of the present invention will also typically include a means for containing the probes, primers, and any other reagent containers in close confinement for commercial sale. Such containers may include injection or blow-molded plastic containers into which the desired vials are retained.

The following examples are included to illustrate various embodiments of the invention. It should be appreciated by those of skill in the art that the techniques disclosed in the examples which follow represent techniques discovered to function well in the practice of the claimed methods. However, those of skill in the art should, in light of the present disclosure, appreciate that many changes may be made in the specific embodiments which are disclosed and still obtain a like or similar result without departing from the spirit and scope of the invention.

### EXAMPLES

### Example 1

In one exemplary method, production of the organic-acid, succinate, in Escherichia coli is disclosed. This exemplary approach may be extended to a broad range of different biorefining applications, including but not limited to, organic acids and other commodities (e.g., ethanol, 1,3 propanediol) and specialty chemicals (e.g., small-molecule drugs, amino-acids).

One recent genomic technology (SCALES) allows rapid identification of genes that alter the growth characteristics of a relevant organism (e.g., bacterial organisms such as E. coli). This approach has been used to identify genes that enhance growth in a variety of different industrially relevant contexts. Improved growth results in improved process productivity, which translates into cheaper manufacturing costs, and biorefining applications, cheaper capital investment costs due to reduced processing requirements. While such applications are clearly of commercial interest, methods for identifying genes to improve product synthesis are also important. A genetic vector that ties succinate concentrations to cell survival and is compatible with the SCALES approach is represented (see FIG. 1). Moreover, this would be the first report of a genomes/vide, ultra high-throughput, product-specific screen for strain engineering applications.

### Biorefining and Organic Acids

### Developing a Tool for SCALES Based High-Throughput Screening

In one exemplary method, the SCalar Analysis of Library Enrichment (SCALES) (Provisional Application No. 60/611,377 filed Sep. 20, 2004 and U.S. patent application Ser. No. 11/231,018 filed Sep. 20, 2005 both entitled: “Mixed-Library Parallel Gene Mapping Quantitation Microarray Technique for Genome Wide Identification of Trait Conferring Genes,” incorporated herein by reference in its entirety) approach allows us to rapidly identify genes for which increased copy number, overexpression, and/or point mutation confers a growth advantage in a relevant environment (see Fig. below). This is a significant advantage over competing technologies where the genetic basis of selected phenotypes most often is unknown, which not only challenges the development of new intellectual property but also any attempts for additional strain improvements. The SCALES approach addresses these issues to produce quantitative, gene-specific data at the genome-scale. The approach combines i) growth selections upon mixtures of plasmid based genomic libraries, ii) Affymetrix gene-chips, and iii) a multi-scale mathematical analysis.

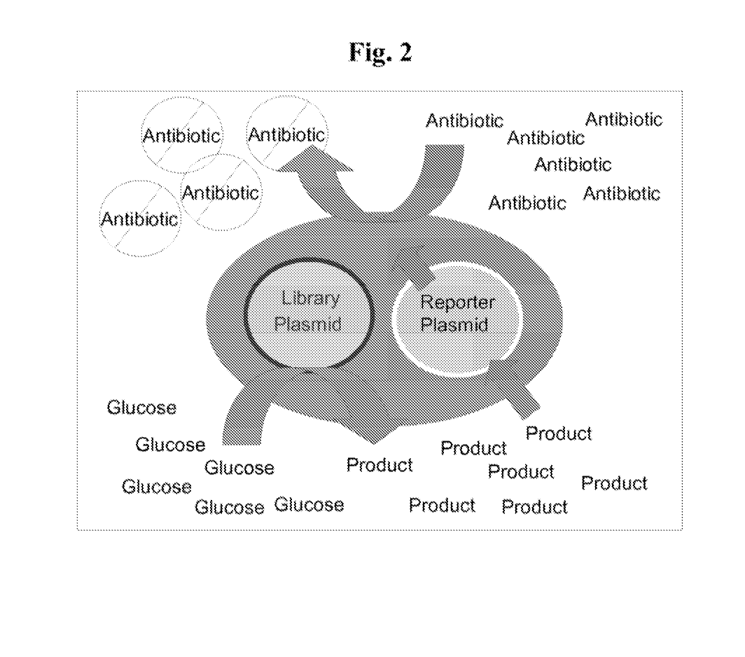

FIG. 1 represents an overview of the SCALEs approach. (a) Genomic DNA fragmented to several specific sizes is ligated into vectors creating several libraries with defined insert sizes. (b) These libraries are individually transformed into the cell line used for selections. (c) The pools of transformants are mixed and subjected to selection. Only clones bearing plasmids with insert increasing fitness survive. (d) Enriched plasmids are purified from the selected population, prepared for hybridization and applied to a microarray. (e) After analyzing the microarray signal, the processed signal is treated as a function of sequence position. (f) A nonlinear multi-scale decomposition gives the signal not only as a function of position but also as a function of scale or library size. The ReSCALEs approach is similar with the exception that the cell line used for selections carries a reporter that links product accumulation to fitness (growth or survival). FIG. 2 illustrates an example of this concept. If a plasmid in the library confers a phenotype of higher production rates or concentrations, this will correspond to a higher activation of the reporter plasmid and a resulting increase in antibiotic tolerance.

This approach may be used in a variety of different contexts including biofilm FORMATION, aerobic/anaerobic growth, and chemical tolerance. Several different genetic loci have been identified for which overexpression increases tolerance to organic-acids. Organic acids including succinate inhibit the growth of E. coli, thus making their production more expensive. Initial SCALEs studies with succinate have led to the identification of several genes that increase E. coli growth in succinate by up to 15%. Analysis of these results led to an understanding that many of the genes that increase succinate tolerance, may do so by degrading succinate, and thus decreasing its concentration in the media. This is a commonly encountered problem in strain engineering-product synthesis and cellular growth are often at odds with one another. As such, techniques that link product levels to cell growth and/or survival are of major importance.

To increase the utility of the SCALEs approach, it is desired to be able to select bacteria that produce more succinate (e.g. more succinate produced=faster growth in an given environment). To do this a genetic system that links succinate production, (extracellular succinate concentration), to survival in E. coli was designed (FIG. 1). The novel feature of this vector is the use of a naturally occurring two component regulatory system specific to succinate. Constitutive expression of this system will be engineered into the plasmid vector. The system responds to succinate by turning on a gene from a particular promoter, dcuBP2. By placing the gene for kanamycin resistance behind this promoter, this vector links the presence of succinate to kanamycin resistance, and thus cell growth. Even though this system is specific to succinate, there are a large variety of such metabolite sensing systems in E. coli. Thus, these technologies will improve the production of a wide-variety of different compounds in various biorefining applications.

A genome-wide strategy linking product levels with cell growth or survival is disclosed herein. Moreover, given the potential for this technology to be adapted to a broad variety of industrial products, additional applications will rapidly follow. As one example, ethanol can be produced by E. coil at relatively high levels using a broad range of inexpensive agricultural waste compounds. One major problem has been that E. coli has relatively low ethanol tolerance compared to yeast or Z. mobilis. Using the approach disclosed herein, a commercial lab might choose to ethanol levels to survival in E. coli and then select for mutants conferring increased growth using) our SCALES approach. Since SCALES generate genome-wide, quantitative growth phenotypes, it would then be possible to identify specific, patentable genes that can be used to improved ethanol production (both growth and concentration) in E. coli. In our initial studies of succinate, we improved growth by 15% through the use of SCALES. Using data from a recent report where fermentation costs were listed as roughly 20% of total ethanol production costs, we estimate that a similar Improvement would reduce total production costs by 3%. Within the context of multi-billion dollar a year products, a 3% reduction in production costs is a considerable savings.

There are however a few issues of using RESCALEs to find increased production of each of the top 12 value-added chemicals or fuels. Currently about half of the top 12 chemicals and none of the potential fuels, have naturally occurring promoters in E coli that could be easily turned into reporter. These include promoters responsive to succinate, fumarate, malate, glycerol and sorbitol. To utilize the ReSCALEs method for other products new promoters are being developed for new reporters. In one embodiment, promoters can be developed by mutating natural promoters that respond to chemically similar compounds. In other embodiments, one general method to evolve new promoters that respond to desired products would be helpful, particularly one that would allow for the evolution of promoters with a more desirable response in relation to product dosage. Below one method is described.

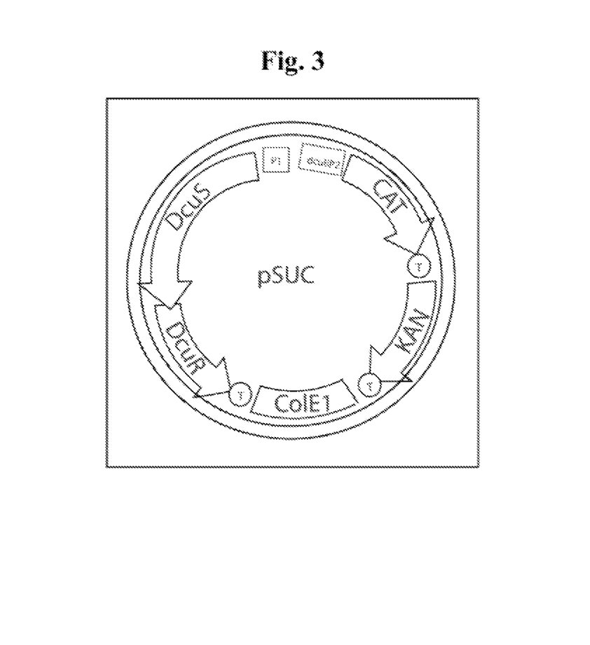

In one exemplary methods, to demonstrate the ReSCALEs approach E coli succinate production strain NZN111 was chosen and a reporter for succinate. Strain NZN111 (Bunch et al., 1997) has deletions in the genes that produce lactate and formate under fermentative anaerobic conditions and as result produces more pure succinate another natural fermentation product. However the production rate of succinate in this strain is very poor. A reporter for succinate was constructed and is illustrated in FIG. 3. Similar reporters can be made for compounds including, but not limited to fumarate, malate, glycerol and sorbitol. This succinate reporter takes advantage of a naturally occurring two-component sensor-regulator in E. coli that responds to external C-4 dicarboxylates including, but not limited to, succinate The reporter termed pSUC contains the two component sensor-regulator genes dcuS and dcuR under the constitutive LacIq promoter as well as the reporter gene (chloramphenicol acetyltransferase in FIG. 3) under the anaerobic dcuB promoter which is activated by the dcuSR system in response to succinate.

### Example 2

pSUC Reporter

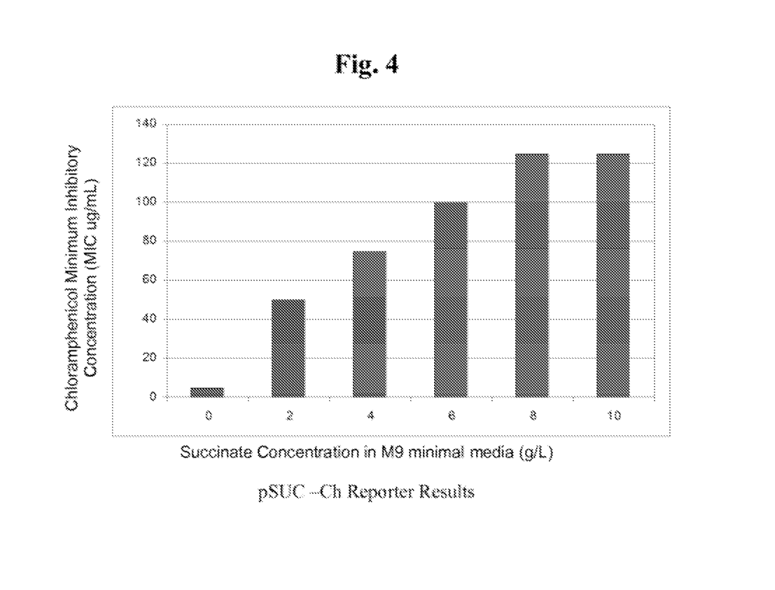

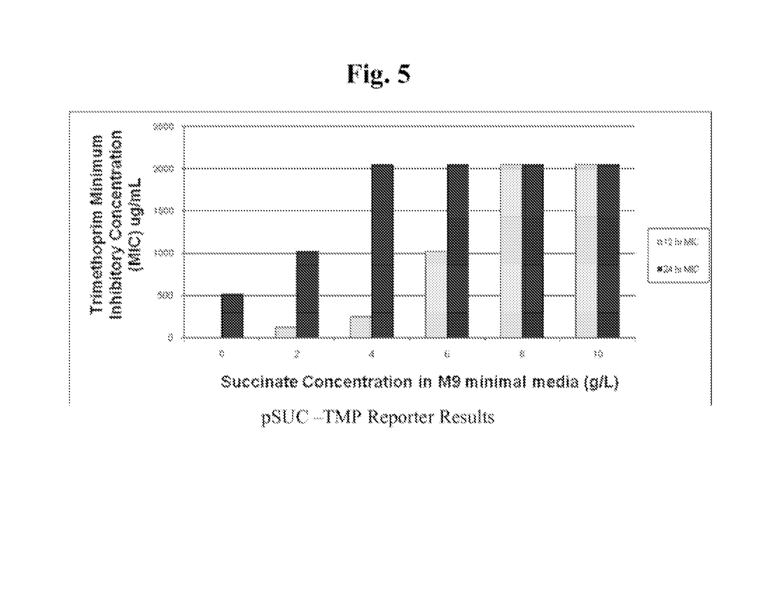

In one exemplary method, two versions of the pSUC reporter plasmid were constructed, pSUC-Ch and pSUC-TMP, the first with a chloramphenicol acetyltransferase (CAT) gene as the reporter and the other with a dihydrofolate reductase gene conferring trimethoprim tolerance as the reporter. E coli K12 with either reporter demonstrated a desired response to external succinate concentrations. (FIGS. 4 and 5). With increasing succinate concentrations E. coli K12+pSUC-Ch and E. coli K12+pSUC-TMp demonstrated dose dependant increases in tolerance to chloramphenicol and trimethoprim, respectively.

### Example 3

pSUC-Ch Reporter

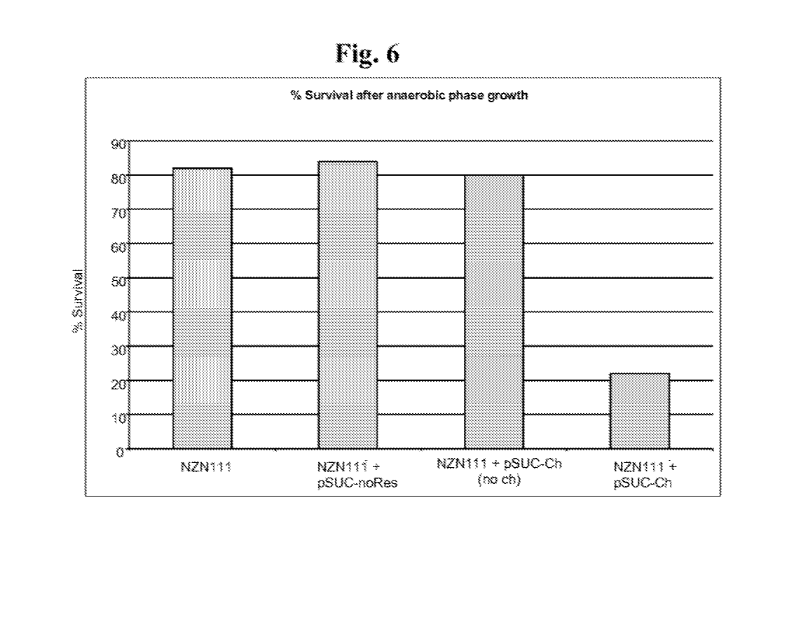

pSUC-Ch is toxic to NZN111 during anaerobic growth fermentations (FIG. 6) This toxicity is due to the chloramphenicol acetyltransferase (CAT) activity and presence of chloramphenicol. This toxicity is specific to NZN111 and is likely a result of acetyl-coA consumption as the strain NZN111 has a defect in acetyl-coA synthesis during anaerobic growth. Acetyl-coA is a necessary substrate for chloramphenicol acetyltransferase and necessary for the detoxification of chloramphenicol. pSUC-Tmp confers tolerance to trimethoprim rather than chloramphenicol, and is not toxic during anaerobic growth. However, since trimethoprim is an antimetabolite inhibiting growth rates rather than exerting a bacteriocidal (killing) effect, selection is very time sensitive, small effects on growth rate in addition to succinate production can have a large affect on fitness of the organism.

### ReSCALEs

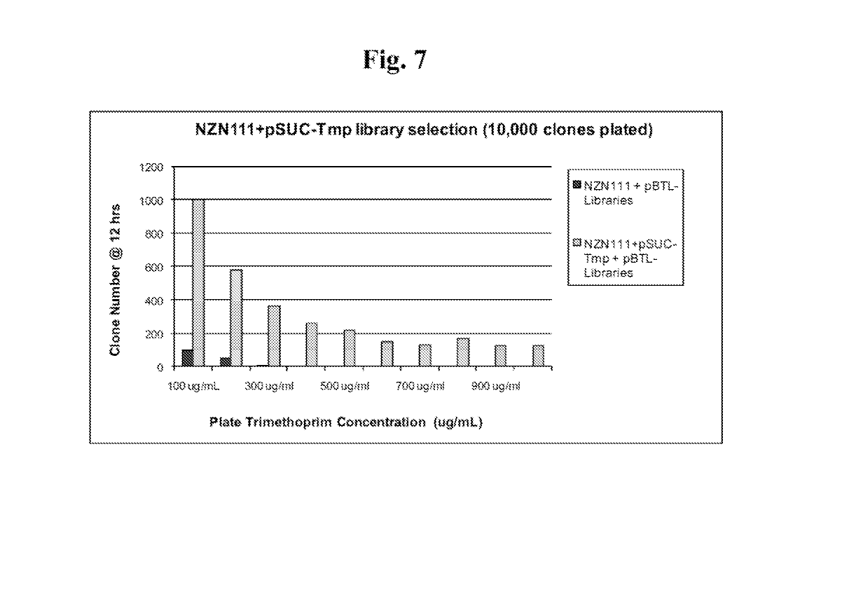

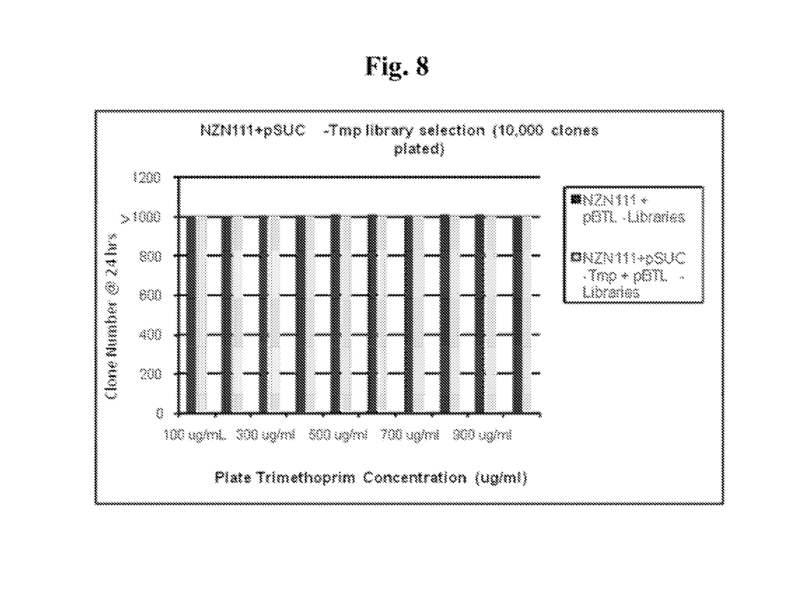

NZN111 bearing the nontoxic pSUC-Tmp reporter was transformed with multiple sized E coli K12 genomic libraries constructed in the pBTL-1 vector (Lynch & GUI, 2006). Libraries included insert sizes of 0.5 kbp, 1 kbp, 2 kbp, 4 kbp and greater than 8 kbp. Libraries contracted in pBTL-1 vector have been shown to have better representation than those constructed in more conventional vectors. Each of these five libraries contained enough clones (10̂5-10̂6 clones) to ensure with 99% probability that the entire genome was represented. 10,000 clones from these libraries (in NZN111+pSUC-TMP) were plated per plate and grown under succinate producing fermentative anaerobic conditions for 12 hours and then allowed to recover aerobically for either 12 (FIG. 7) or 24 hrs (FIG. 8). After 12 hours of recovery there were numerous clones with higher growth rates on higher concentrations of trimethoprim with the genomic libraries when compared to the control indicating numerous clones activating the reporter presumably with higher surrounding succinate levels. This difference disappeared at 24 hours of recovery indicating that the growth rate of the control strain even at high levels of trimethoprim was high enough to take over the plates at 24 hours.

Bacteria, plasmids, media and library construction. Wild-type Escherichia coli K12 (ATCC #29425) for the preparation of genomic DNA were used. Cultures were grown for library construction in Luria-Bertani (LB) medium at 37 1 C. Genomic libraries were constructed of insert sizes 500, 1,000, 2,000, 4,000 and 48,000 base pairs of E. coli strain K12 genomic DNA in the pBTL-1 vector (low-copy) according to standard methods. Greater than 10⁶ and 10⁵ clones for libraries with insert sizes less than or equal to 4,000 base pairs or greater than 8,000 base pairs, respectively were obtained. The anaerobic succinate producing strain, Escherichia coli NZN111 was used for fermentations and for library studies.

Reporter Construction: Standard molecular biological techniques were used for plasmid construction including routing protocols for polymerase chain reaction, gene synthesis, and molecular cloning. The pSUC reporter plasmids were constructed as follows: A small genetic element containing the constitutive lacIq promoter oriented in one direction and the dcuBP2 promoter in the other direction was synthesized by gene synthesis and inserted into the pSMART-HC-KAN vector from Lucigen (Middleton, Wis.) creating the plasmid pS1. The dcuSR operon was amplified from E. coli genomic DNA by polymerase chain reaction and inserted downstream of the lacIq promoter of pS1 creating pSUC. The chloramphenicol acetyltransferase (CAT) gene or dihydrofolate reductase gene (DHFR) gene were similarly amplified from pBT-3 and pBT-5, respectively (previously reported) and inserted downstream of the dcuBP2 promoter of pSUC to create the two reporter plasmids pSUC-Ch and pSUC-TMP.

Reporter Assays. E coli K12 was transformed with the reporter vectors pSUC-Ch or pSUC-TMP and grown overnight in LB media and then used to inoculate (10% by volume) 96 well plates containing 100 mL of M9 minimal media supplemented with various concentrations of succinate. These plates were incubated at 37° C. in Type A anaerobic BioBags (Fisher Scientific).

Survival Challenges. NZN111, NZN111+pSUC and NZN111+pSUC-Ch were grown overnight on LB agar plates or LB agar plates containing chloramphenicol 100 ug/ml at 37° C. in either Type A anaerobic Bio Bags or in air.

Library Selections: NZN111+pSUC-TMP and NZN111 were made competent by standard methods and transformed with the 5 E. coli K12 genomic libraries in pBTL-1. Greater than 10⁶ and 10⁵ clones for libraries with insert sizes less than or equal to 4,000 base pairs or greater than 8,000 base pairs, respectively were obtained. 10,000 clones of NZN111+pSUC-TMP+Libraries or NZN111 +libraries were plated on LB agar plates with various concentrations of trimethoprim and incubated 37 degrees C. either in Type A anaerobic BioBags or in air.

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method comprising:
a) obtaining a chemical reporter having a set of genetic elements comprising, a chemical sensor module; a regulated promoter; and a selectable marker; and
b) using the chemical reporter with a multi-scale analysis library enrichment method (SCALEs) to identify a genetic element capable of altering the expression of a non-selectable phenotype.

2. The method of claim 1, wherein the genetic elements comprise a vector.

3. The method of claim 2, wherein, the vector is a plasmid.

4. The method of claim 1, wherein the chemical sensor module senses an organic compound.

5. The method of claim 1, wherein the chemical sensor module senses an organic compound selected from the group consisting of succinate, fumaric and malic acids, 2,5 furan dicarboxylic acid, 3-hydroxy propionic acid, aspartic acid, glucaric acid, glutamic acid, itaconic acid, levulinic acid, 3-hydroxybutyrolactone, glycerol, sorbitol, xylitol/arabinitol, and lactate, butanol, n-hexanol, n-octanol, n-heptanol, n-pentanol, n-butanol, propanol, ethanol, isopropanol, isobutanol, pentanol, isopentanol, sec-butanol, 2-pentanol, 2-hexanol, 4-methyl-pentanol and a combination thereof.

6. The method of claim 1, wherein the chemical sensor module regulates expression from the regulated promoter in response to the presence of the organic compound.

7. The method of claim 4, further comprising identifying a microorganism capable of producing increased quantities of the organic compound.

8. A method comprising:
a) generating a vector having a selectable marker, a chemical sensor module and a regulated promoter;
b) using the vector to assess the survivability of a bacterial strain; and
c) selecting mutant libraries bacterial strain based on survivability, wherein the survivability is indicative of a genetic modification of the strain.

9. The method of claim 7, further comprising using survivability of the strain to genetically select for improved survival of the strain.

10. A method for enhanced production of a compound in a host cell comprising:
a. optimizing a single expression regulatory sequence for producing a compound in the host cell utilizing at least two or more nucleic acid constructs;
b. mobilizing the single expression regulatory sequence into a chemical sensor nucleic acid construct and transforming the host cell with the chemical sensor nucleic acid construct;
c. screening for enhanced production of the compound in the host cell by providing a genomic library of predetermined sized genomic sequences, wherein the sequences are transformed in the host cell and one or more of the sequences are associated with the enhanced production;
d. determining the host cell in (c) that provides the enhanced production; and
e. culturing the host cell in (d) to provide the compound.

11. The method of claim 10, wherein the nucleic acid construct(s) are episomal or integrated into the host cell's genome.

12. A host cell comprising a nucleic acid construct comprising a genetic element for increasing production of a compound in a host cell and a second expression regulatory element comprising a chemical sensor module, wherein the chemical sensor module is responsive to the compound.

13. A population of host cells comprising a nucleic acid construct comprising a genetic element that increases the production of a compound in a host cell, and a second nucleic acid construct comprising a chemical sensor module, wherein the chemical sensor module is responsive to the compound, and wherein the population comprises a genomic library of predetermined sized sequences.
